(19) United States
(12) Patent Application Publication
(10) Pub. No.: US 2004/0192772 A1
(43) Pub. Date: Sep. 30, 2004

(54) Agent for treating the symptoms of dementia disorders and/or depression

(76) Inventor: Lothar Saiger, Duermentingen (DE)

Correspondence Address: Kohler Schmid & Partner, Ruppmannstrasse, 27, Stuttgart, D-70565, DE

(21) Appl. No.: 10/766,537

(22) Filed: Jan. 28, 2004

Related U.S. Application Data
(63) Continuation of application No. PCT/DE01/02869, filed on Jul. 31, 2001.
(63) Continuation of application No. PCT/DE01/02870, filed on Jul. 31, 2001.

(51) Int. Cl. A61K 31/24

(52) U.S. Cl. 514/534

(57) ABSTRACT

An agent for producing medication for the treatment of depression and/or dementia disorders improves the effectivity of treatment of this illness compared to classical therapy through a combination of a substance which increases the dopamine concentration in the synaptic gap of the nerve cells of the brain and a local anaesthetic of the anilide group.

This application is a continuation of PCT/DE01/02869 and of PCT/DE01/02870 both filed on Jul. 31, 2001. The entire disclosures of both are hereby incorporated by reference.

### BACKGROUND OF THE INVENTION

The invention concerns an agent for producing medication for the treatment of the symptoms of dementia disorders or dementia and/or for the treatment of depression.

Dementia is a general brain disease, wherein, in particular, regions of the brain are also damaged which are responsible for the regulation of the function of the other brain regions, in particular of the cortex.

Dementia disorders such as brain arteriosclerosis, Alzheimer disease or the Pick disease are characterized in that normal mental performance is no longer possible, since a plurality of different brain regions are required for solving a predetermined task and these brain regions must be activated and must communicate with each other for this purpose. To sum up 2+3, one brain region must be activated in which the numbers are stored, a further brain region must be activated which recognizes the links and functional context and where functions such as addition and subtraction are stored, and one further brain region must be activated which recognizes the result and associates it with the number region.

One characteristic of dementia is, in particular, the fact that the communication between different brain regions decreases or is completely absent. For this reason, initially more complex, and at an advanced stage, even simple tasks can no longer be performed.

Dementia disorders are generally recognized by the concerned individuals themselves as a deterioration of their own brain performance and this causes great suffering.

Conventional improvement of the brain performance of persons suffering from dementia is successful only to a limited degree.

Depression is generally caused through decrease of the biochemical transmitting agents or transmitters or neurotransmitters such as dopamine, noradrenaline and serotonine.

The concerned individuals themselves experience depression as a feeling of general powerlessness, wherein this feeling is caused consciously or unconsciously by a limitation in their individual brain performance.

If the person concerned does not consciously experience his/her limited brain performance, he/she still will perceive a general powerlessness compared to other people or compared to their own earlier phases in life. This produces the feeling of not being able to structure their lives, wherein a life controlled by others is again experienced as having little sense and substantially not worth living.

If, however, a person concerned consciously perceives his/her limitation of brain performance, he/she will be disturbed upon noticing his/her own reduced activity or capability of activation of the brain, and he/she will often misinterpret his/her environment and not be able to structure it in a positive manner in consequence of this malfunction, and will rather feel like a plaything of frightening, unfathomable confusing powers compared to persons who are able to give their environment a positive structure. Under such circumstances, these persons will perceive their own lives as having little sense and substantially not worth living.

The cause of the underlying subjective emotional mood of persons suffering from depression is therefore the feeling or recognition of a lack of control of the environment or their own powerlessness and the resulting lack of importance or superfluousness of themselves. The cause thereof, i.e. the limitation of their own brain performance is actually individually perceived by only a few patients.

It is therefore the underlying purpose of the invention to provide an agent for the effective treatment of dementia disorders and the symptoms of dementia disorders and/or depression and/or the symptoms of depression.

### SUMMARY OF THE INVENTION

In accordance with the invention, this object is achieved by a local anaesthetic of the anilide group.

This object is achieved in particular by combined application of

a substance increasing the dopamine concentration in the synaptic gap of the nerve cells of the brain; and

a local anaesthetic of the anilide group or its derivatives.

Preferred embodiments of the invention are the subject matter of the dependent claims.

### DESCRIPTION OF THE PREFERRED EMBODIMENT

In accordance with a preferred embodiment of the inventive agent, the local anaesthetic of the anilide group is the substance mepivacaine, preferably in a daily dose of 30 mg to 60 mg. As an alternative for mepivacaine, the substances lidocaine, bupivacaine, butanilicaine, tholycaine or etidocaine may be used.

In the inventive agent, the combination of a substance increasing the dopamine concentration in the synaptic gap of the nerve cells of the brain, and a local anaesthetic of the anilide group or its derivatives leads to quintessential activation of the inter-cellular combination of the cerebral nerve cells of a person suffering from dementia and/or depression. The same is true for the treatment of persons suffering from depression. The corresponding explanation is given below. Due to this effect, the roots of dementia disorders can be treated and not only their symptoms. The function of the inventive agent is based on the following findings:

Two important brain regions which are responsible for the regulation of the function of the other brain regions, in particular of the cortex, are the so-called parasympathetic system and the sympathetic system.

Dementia can therefore be traced back, in particular, to a malfunction of the parasympathetic system and/or the sympathetic system of the human brain.

The parasympathetic system controls the processes in the body which build up energy such as sleep, digestion and relaxation. It reduces the blood pressure, the pulse rate and converts glucose into glycogen. The neurotransmitter in the parasympathetic system is mainly serotonine.

The sympathetic system controls the processes requiring energy such as heart activation, blood pressure increase and blood sugar mobilization. The neurotransmitter in the sympathetic system is mainly noradrenaline.

There is an empirically established relationship between the concentration of dopamine and the concentration of serotonine. There is also a relationship between the concentration of dopamine and the concentration of serotonine and noradrenaline.

In the case of dementia, in particular, the supply of dopamine, noradrenaline and serotonine in the brain is generally disturbed. For this reason, sufficient concentration of these substances which is required for normal brain function is not ensured.

In the case of depression, the neurotransmitter serotonine plays an important role. Serotonine is the so-called pleasure hormone, wherein a person will feel happy or at least not feel depressed when a predetermined concentration of serotonine is present in the brain. There is an empirical relationship between the serotonine concentration and the dopamine concentration in the brain.

Depression can be traced back, in particular, to a malfunction of the so-called parasympathetic system and/or sympathetic system of the human brain.

Also in case of these symptoms, the parasympathetic system controls the processes in the body which build up energy such as sleep, digestion and relaxation. It reduces the blood pressure, the pulse rate and converts glucose into glycogen. The neurotransmitter in the parasympathetic system is mainly serotonine. The sympathetic system controls the processes requiring energy such as heart activation, blood pressure increase and blood sugar mobilization. The neurotransmitter in the sympathetic system is mainly noradrenaline.

It has been empirically proven that there is a relationship between the concentration of dopamine and the concentration of serotonine. The higher the concentration of dopamine in the brain, the higher the concentration of serotonine and noradrenaline.

For persons suffering from depression in general, the supply of dopamine, noradrenaline and serotonine in the brain is particularly disturbed. For this reason, the sufficient concentration of these substances, which is required for normal brain function, is not guaranteed.

Supply of sufficient dopamine, noradrenaline and serotonine concentrations in the brain, which is required for normal brain function, can be provided in accordance with the invention through precise application of a local anaesthetic of the anilide group. Application thereof increases the permeability of the blood/brain barrier such that more L-Dopa can pass this barrier and enter into the brain. L-Dopa is a substance which is converted in the Substantia Nigra, a further brain region, into dopamine. Dopamine is a precursor for forming serotonine and noradrenaline. Administration of dopamine induces an associated increase in the serotonine concentration and positively influences formation of glycogen in the parasympathetic system, i.e. more glycogen is emitted which is provided to the body for processes requiring energy. At the same time, an increase in the noradrenaline concentration induced by application of dopamine in the sympathetic system causes more glycogen to be released to be used in the muscles, since more glycogen is converted into glucose which is directly provided, through the blood stream, to the muscle and also, in particular, to the nerve system of the brain.

Supply of sufficient dopamine, noradrenaline and serotonine concentrations in the brain which is required for normal brain function through administration of the inventive agent, causes positive changes in the conductivity of the intercellular nerve connections in the synaptic gap such that signal transmission from one nerve cell to the next is improved. The inventive agent thereby increases the transmission potential of nerve cell connections, in particular, in the brain.

General activation of the brain potential through additional emission of glucose caused by increased formation of glycogen in the parasympathetic system, and conversion of the glycogen into glucose in the sympathetic system, considerably improves the general symptoms of the various dementia disorders.

In the inventive agent, the combination of a substance which increases the dopamine concentration in the synaptic gap of the nerve cells of the brain, with a local anaesthetic of the anilide group causes an increase in the permeability of the blood-brain barrier for the substance LevoDopa such that dopamine can be deposited in a higher concentration in the brain of persons suffering from dementia disorders or depression compared to conventional standard therapies, with the result that the concentration of dopamine in the brains of these persons is increased.

The following fact should be noted:

In particular, dopamine is not suited for passing the blood/brain barrier under normal conditions, i.e. without simultaneous presence of a local anaesthetic of the anilide group. Instead of dopamine, L-Dopa must be administered as standard, since it is able, in contrast to dopamine, to pass the blood/brain barrier with a certain, however, lower percentage, without the presence of a local anaesthetic of the anilide group. L-Dopa is a substance which is converted into dopamine in the Substantia Nigra, a part of the brain. However, this conversion requires a functioning Substantia Nigra whose function, like that of other brain regions, is ensured only when a sufficient amount of dopamine is present. For this reason, the classical standard therapy of dementia disorders fails at a certain point in the development of this disorder due to insufficient supply of dopamine to the brain. This point can be overcome by a therapy which provides direct application of dopamine with simultaneous application of an agent for increasing the permeability of the blood/brain barrier for dopamine, such that the dopamine concentration in the brain can be directly influenced and insufficient supply can be compensated for even for advanced diseases. These findings can be transferred to the symptoms of persons suffering from depression.

The inventive substance “local anaesthetic of the anilide group” belongs generally to local anaesthetics of varying structure, wherein the local anaesthetic of the anilide group and its derivatives, a subgroup of these local anaesthetics, are preferably used for therapy. In addition to mepivacaine, embodiments of this subgroup include lidocaine, bupivacaine, butanilicaine, etidocaine, tholycaine and ropivacaine. Mepivacaine has the smallest molecule in the group and has proven to be the most effective for the therapy of patients suffering from dementia disorders and depression. One assumes that, due to the small molecular size of mepivacaine, the probability of passage to the blood/brain barrier is higher. Mepivacaine is also lipophilic, i.e. fat-loving, and tends to join fat molecules. In this connection it must be noted that nerve cells are mostly embedded in fat and provision or enrichment of mepivacaine in fat should also have an effect on the nerve paths extending through fatty tissue. Like mepivacaine, LevoDopa is also highly lipophilic and this realization could lead to the activating mechanism.

In the inventive agent for producing medication for treatment of dementia disorders, LevoDopa is preferably applied in a daily dose of 200 mg to 600 mg.

In accordance with an alternative embodiment of the inventive agent, the substance which increases the dopamine concentration in the synaptic gap of the nerve cells of the brain additionally contains bromocriptine, which is preferably applied in a daily dose of 1.25 mg to 10 mg.

In accordance with a further embodiment of the inventive agent, the substance which increases the dopamine concentration in the synaptic gap of the nerve cells of the brain additionally contains selegiline, which is preferably applied in a daily dose of 4 mg to 20 mg.

In accordance with a further alternative embodiment of the inventive agent, the substance which increases the dopamine concentration in the synaptic gap of the nerve cells of the brain additionally contains amantadine, which is preferably applied in a daily dose of 100 mg to 400 mg.

In accordance with a further alternative embodiment of the inventive agent, the substance which increases the dopamine concentration in the synaptic gap of the nerve cells of the brain additionally contains pergolide mesilate, which is preferably applied in a daily dose of 2 mg to 8 mg. In accordance with another embodiment, the inventive agent may also contain tolcapone as a substance which increases the dopamine concentration in the synaptic gap of the nerve cells of the brain, which is applied in a daily dose of 100 mg to 400 mg.

In accordance with another inventive embodiment of the inventive agent, the substance which increases the dopamine concentration in the synaptic gap of the nerve cells of the brain could additionally contain piracetam, which is applied in a daily dose of 1000 mg to 4000 mg.

The substances mentioned above which increase the dopamine concentration in the synaptic gap of the nerve cells of the brain, may be contained in the inventive agent in accordance with the invention individually and in various combinations. The effect of the inventive agent is based less on a special combination of substances of classical Parkinson therapy, which increase the dopamine concentration in the synaptic gap of the nerve cells of the brain, but rather on a combination of these substances used for classical Parkinson therapy with a local anaesthetic, in particular, a local anaesthetic of the anilide group and thereby in particular, but not exclusively with the substance mepivacaine.

The above-mentioned doses of the local anaesthetics refer to injected applications. For oral application, the dose must be correspondingly adapted.

I claim:

1. A use of an active substance for producing medication to treat dementia disorders and/or depression, the substance comprising:
means for increasing a dopamine concentration in a synaptic gap of brain nerve cells, and
an anilide group local anaesthetic or a derivative thereof.

2. The use of claim 1, wherein mepivacaine is said anilide group local anaesthetic or said derivative thereof.

3. The use of claim 2, wherein said mepivacaine is applied in a daily dose of 30 mg to 60 mg.

4. The use of claim 1, wherein lidocaine is said anilide group local anaesthetic or said derivative thereof.

5. The use of claim 4, wherein said lidocaine is applied in a daily dose of up to 150 g.

6. The use of claim 1, wherein bupivacaine is said anilide group local anaesthetic or said derivative thereof.

7. The use of claim 6, wherein said bupivacaine is applied in a daily dose of up to 150 mg.

8. The use of claim 1, wherein butanilicaine is said anilide group local anaesthetic or said derivative thereof.

9. The use of claim 1, wherein tholycaine is said anilide group local anaesthetic or said derivative thereof.

10. The use of claim 1, wherein etidocaine is said anilide group local anaesthetic or said derivative thereof.

11. The use of claim 1, wherein ropivacaine is said anilide group local anaesthetic or said derivative thereof.

12. The use of claim 11, wherein said ropivacaine is applied in a dose of 0.2 mg to 4 mg.

13. The use of an agent for producing medication for treatment of dementia disorders and/or depression according to claim 1, wherein said means for increasing said dopamine concentration in said synaptic gap of said brain nerve cells contains LevoDopa.

14. The use of claim 13, wherein said LevoDopa is applied in a daily dose of 200 mg to 600 mg.

15. The use of claim 13, wherein said means for increasing said dopamine concentration in said synaptic gap of said brain nerve cells additionally contains bromocriptine.

16. The use of claim 15, wherein said bromocriptine is applied in a daily dose of 0.1 mg to 10 mg.

17. The use of claim 13, wherein said means for increasing said dopamine concentration in said synaptic gap of said brain nerve cells additionally contains selegiline.

18. The use of claim 17, wherein said selegiline is applied in a daily dose of 4 mg to 20 mg.

19. The use of claim 18, wherein said means for increasing said dopamine concentration in said synaptic gap of said brain nerve cells additionally contains amantadine.

20. The use of claim 19, wherein said amantadine is applied in a daily dose of 100 mg to 400 mg.

21. The use of claim 13, wherein said means for increasing said dopamine concentration in said synaptic gap of said brain nerve cells additionally contains pergolide mesilate.

22. The use of claim 21, wherein said amantadine is applied in a daily dose of 2 mg to 8 mg.

23. The use of claim 22, wherein said means for increasing said dopamine concentration in said synaptic gap of said brain nerve cells additionally contains tolcapone.

24. The use of claim 23, wherein said tolcapone is applied in a daily dose of 100 mg to 400 mg.

25. The use of claim 13, wherein said means for increasing said dopamine concentration in said synaptic gap of said brain nerve cells additionally contains piracetam.

26. The use of claim 25, wherein said piracetam is applied in a daily dose of 1,000 mg to 4,000 mg.
